(19) United States
(12) Patent Application Publication
(10) Pub. No.: US 2013/0066150 A1
(43) Pub. Date: Mar. 14, 2013

(54) ZOOM LENS MODULE AND ENDOSCOPE SYSTEM INCLUDING THE SAME

(71) Applicants: Seung-wan LEE, Suwon-si (KR); Yeon-ho KIM, Hwaseong-si (KR); Min-seog CHOI, Seoul (KR); Woon-bae KIM, Seoul (KR)

(72) Inventors: Seung-wan LEE, Suwon-si (KR); Yeon-ho KIM, Hwaseong-si (KR); Min-seog CHOI, Seoul (KR); Woon-bae KIM, Seoul (KR)

(73) Assignee: SAMSUNG ELECTRONICS CO., LTD., Suwon-si (KR)

(21) Appl. No.: 13/532,198

(22) Filed: Jun. 25, 2012

(30) Foreign Application Priority Data
Sep. 9, 2011 (KR) 10-2011-0092227

(51) Int. Cl. A61B 1/002; A61B 1/06; G02B 26/02; G02B 3/14; G02B 27/42

(52) U.S. Cl. 600/163; 359/666; 359/558; 359/228

(57) ABSTRACT

A zoom lens module and an endoscope system including the zoom lens module are disclosed. The zoom lens module includes: a first liquid lens; a second liquid which is disposed separated from the first liquid lens; and an aperture disposed between the first and second liquid lenses. A respective focal distance of each of the first liquid lens and the second liquid lens is adjustable based on a change of at least one of the respective curvature thereof and the respective thickness thereof.

### CROSS-REFERENCE TO RELATED APPLICATIONS

This application claims priority from Korean Patent Application No. 10-2011-0092227, filed on Sep. 9, 2011, in the Korean Intellectual Property Office, the disclosure of which is incorporated herein by reference in its entirety.

### BACKGROUND

1. Field

The present disclosure relates to a zoom lens module and an endoscope system employing the same.

2. Description of the Related Art

Recently, endoscopic surgery is becoming increasingly popular due to its typical effects of reduced surgical wounds and fast recovery of patients. In conjunction with an increasing dependence on the use of surgical robots for performing surgery on small body glands, such as the prostate gland, the thyroid gland, and the like, the use of 3-dimensional stereoscopic endoscope systems or surgical robots are expected to offer surgeons a more accurate distance perception and to prevent shaking of a surgeon's hand during an operation.

In a practical operation that uses an endoscope system, if a captured image is not sharp, a body part to be excised and a healthy body part to remain may look indistinguishable, and the healthy body part is likely to be removed by mistake. Therefore, to assist surgeons to selectively remove only damaged body parts, there is an increasing demand for an endoscope system that provides sharp, clear stereoscopic images and an optical zooming function.

### SUMMARY

Provided is a small zoom lens module that uses at least one liquid lens.

Provided is an endoscope system which includes the zoom lens module.

Additional aspects will be set forth in part in the detailed description which follows and, in part, will be apparent from the detailed description, or may be learned by practice of the presented exemplary embodiments.

According to an aspect of one or more exemplary embodiments, a zoom lens module includes: a first liquid lens; a second liquid lens disposed separated from the first liquid lens; and an aperture disposed between the first and second liquid lenses. A respective focal distance of each of the first liquid lens and the second liquid lens is adjustable based on a change of at least one of a respective curvature thereof and a respective thickness thereof.

The zoom lens module may further include a diffractive optical element (DOE) lens array disposed at least one of between the first liquid lens and the aperture, and between the aperture and the second liquid lens.

The zoom lens module may further include a dielectric layer on the DOE lens array.

At least one of the first and second liquid lenses may have a curvature radius which is less than or approximately equal to 2.5 mm.

An interval between the first liquid lens and the second liquid lens may have a length which is less than or approximately equal to 2.5 mm.

At least one of the first and second liquid lenses may include: a first lens fluid; a second lens fluid that is immiscible with the first lens fluid; a first lens chamber which contains the first lens fluid and the second lens fluid; a first surface which functions as an interface between the first lens fluid and the second lens fluid to form a lens surface; a second surface which functions as an interface between the first lens fluid and the second lens fluid that facilitates a change in a curvature of the lens surface; and a first lens electrode unit which shifts a position of the second surface to effect the change in the curvature of the lens surface.

Each of the first lens fluid and the second lens fluid may be light-transmissive.

The zoom lens module may further include a first intermediate lens substrate provided in the first chamber, the first intermediate lens substrate including a first through-hole which defines a diameter of a lens corresponding to the lens surface, and a second through-hole which defines a path of the second lens fluid.

The zoom lens module may further include: a first lower lens substrate disposed below the first intermediate lens substrate; a first upper lens substrate disposed above the first intermediate lens substrate; and a first spacer unit disposed between the first lower lens substrate and the first intermediate lens substrate, and a second spacer unit disposed between the first intermediate lens substrate and the first upper lens substrate.

The first lens electrode unit may include at least one electrode coated with an insulating material.

The aperture may include: a first aperture fluid; a second aperture fluid that is immiscible with; a first aperture chamber which contains the first aperture fluid and the second aperture fluid; and a first aperture electrode unit which adjusts a size of an opening through which light passes by shifting a position of an interface between the first aperture fluid and the second aperture fluid. One of the first aperture fluid and the second aperture fluid is light-transmissive, and an other of the first aperture fluid and the second aperture fluid is formed of a light-blocking material.

The first aperture chamber may include: a channel region which corresponds to a range of the size of the opening that is adjustable by changing the position of the interface between the first aperture fluid and the second aperture fluid; and a reservoir region which stores each of the first and second aperture fluids to move into the channel region based on a shift in the position of the interface between the first aperture fluid and the second aperture fluid.

The first aperture chamber may include: a first lower aperture substrate which contains the first aperture electrode unit; a first intermediate aperture substrate disposed facing toward and separated from the first lower aperture substrate; and a first upper aperture substrate disposed facing toward and separated from the first intermediate aperture substrate.

The first intermediate aperture substrate may include a through-hole in a center region thereof.

The one of the first aperture fluid and the second aperture fluid that is light-transmissive may be provided in a center region of the first aperture chamber, and the other of the first aperture fluid and the second aperture fluid that is formed of the light-blocking material may be provided in a peripheral region of the first aperture chamber which peripheral region surrounds the center region.

The first aperture chamber may include: a first channel; and a second channel disposed on the first channel, the second channel being interconnected with the first channel, wherein a range of the size of the opening may be defined by a corresponding range of shifts in the position of the interface between the first aperture fluid and the second aperture fluid within each of the first and second channels.

According to another aspect of one or more exemplary embodiments, an endoscope system includes: an illumination light providing unit which provides illumination light to a target; an imaging unit which captures an image of the target; and a light transmission unit which includes any one of the zoom lens modules described above, and which transmits the illumination light to the target and which transmits light reflected from the target to the imaging unit.

The endoscope system may further include an insertion unit within which the light transmission unit is installed and which is insertable into a body cavity.

The light transmission unit may include a waveguide.

The light transmission unit may include: a first light transmission module which transmits the illumination light to the target; and a second light transmission module which includes the zoom lens module and which transmits the light reflected from the target to the imaging unit.

The imaging unit may include: a first imaging unit which captures at least a first parallax image of the target; and a second imaging unit which is disposed separated from the first imaging unit and which captures at least a second parallax image of the target. The at least first parallax image and the at least second parallax image are used for creation of at least one three-dimensional image.

The light transmission unit may include: a first light transmission module which includes the zoom lens module and which transmits a first part of the light reflected from the target to the first imaging unit; and a second light transmission module which includes the zoom lens module and which transmits a second part of the light reflected from the target to the second imaging unit.

At least one of the first and second light transmission modules may include at least one curved region in which a reflecting unit for reflecting light incident on the curved region is disposed.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a schematic view which illustrates a structure of a zoom lens module, according to an exemplary embodiment;

FIGS. 2A, 2B, and 2C are cross-sectional views which illustrate a schematic structure of a first liquid lens used in the zoom lens module of FIG. 1, according to an exemplary embodiment;

FIG. 3 s a schematic cross-sectional view which illustrates a structure of an aperture of the zoom lens module of FIG. 1;

FIGS. 4A and 4B illustrate an adjustment of the light transmission of the aperture of FIG. 3 based on respective different sizes AD1 and AD2 of an opening;

FIG. 5 illustrates an aperture which includes a diffractive optical element (DOE) lens array, according to an exemplary embodiment;

FIG. 6 is a schematic illustration of an optical arrangement in an endoscope system, according to an exemplary embodiment; and

FIG. 7 is a schematic illustration of an optical arrangement in an endoscope system which is configured for capturing three-dimensional images, according to another exemplary embodiment.

### DETAILED DESCRIPTION

Reference will now be made in detail to exemplary embodiments, examples of which are illustrated in the accompanying drawings, wherein like reference numerals refer to like elements throughout, and the thicknesses of layers and regions are exaggerated for clarity. In this regard, the present exemplary embodiments may have different forms and should not be construed as being limited to the descriptions set forth herein. Accordingly, the exemplary embodiments are merely described below, by referring to the figures, to describe various aspects of the present disclosure.

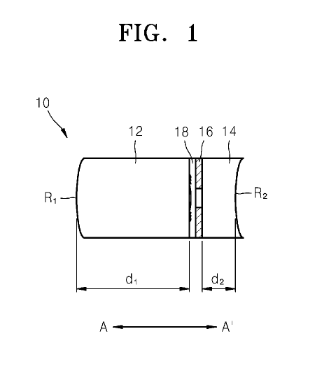

FIG. 1 is a schematic view which illustrates a structure of a zoom lens module 10, according to an exemplary embodiment. Referring to FIG. 1, the zoom lens module 10 includes first and second liquid lenses 12 and 14 that have different respective refractive indices. The first and second liquid lenses 12 and 14 may be disposed apart from each other. An aperture 16 and a diffractive optical element (DOE) lens array 18 may be disposed between the first and second liquid lenses 12 and 14.

A respective curvature and a respective thickness of each of the first and second liquid lenses 12 and 14 may be adjusted independently with respect to the other liquid lens. By selectively adjusting the variable curvature and/or thickness of each of the first and second liquid lenses 12 and 14, a focal distance of the zoom lens module 10 may be adjusted. The focal distance (f) of the zoom lens module 10, which depends on both of the first and second liquid lenses 12 and 14, is represented by Equation 1 below.

$$\frac{1}{f} = ( {n}_{1} - 1 ) ( {n}_{2} - 1 ) \{ \frac{1}{{R}_{1} ( {n}_{2} - 1 )} - \frac{1}{{R}_{2} ( {n}_{1} - 1 )} + ( \frac{{d}_{1}}{{n}_{1}} + \frac{{d}_{2}}{{n}_{2}} ) \frac{1}{{R}_{1} {R}_{2}} \} \qquad [ Equation 1 ]$$

wherein n₁ is a refractive index of the first liquid lens 12, n₂ is a refractive index of the second liquid lens 14, R₁ is a curvature radius of the first liquid lens 12, R₂ is a curvature radius of the second liquid lens 14, d₁ is a thickness of the first liquid lens 12, and d₂ is a thickness of the second liquid lens 14.

The thickness d₁ of the first liquid lens 12 may be at least double the thickness d₂ of the second liquid lens 14. In the zoom lens module 10, which may be installed in an endoscope system, a respective curvature radius of each of the first and second liquid lenses 12 and 14 may be less than or approximately equal to 2.5 mm. Furthermore, a greater distance, or interval, between the first and second liquid lenses 12 and 14 may entail a correspondingly larger zoom lens module 10. To avoid this, the length of the gap between the first liquid lens 12 and the second liquid lens 14 may be less than or approximately equal to 2.5 mm.

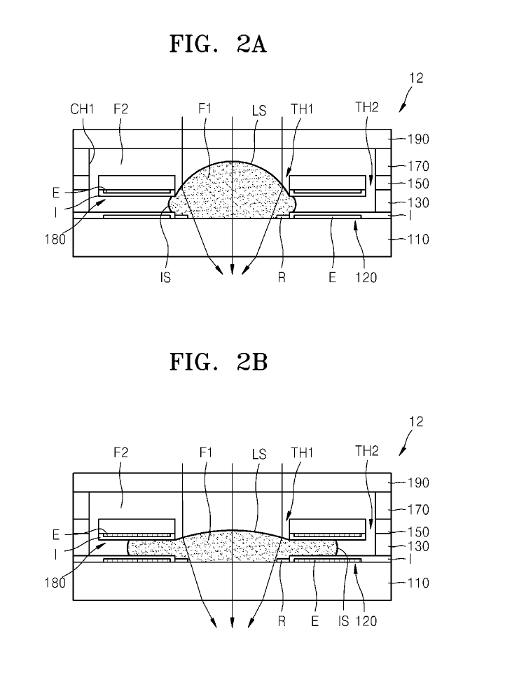

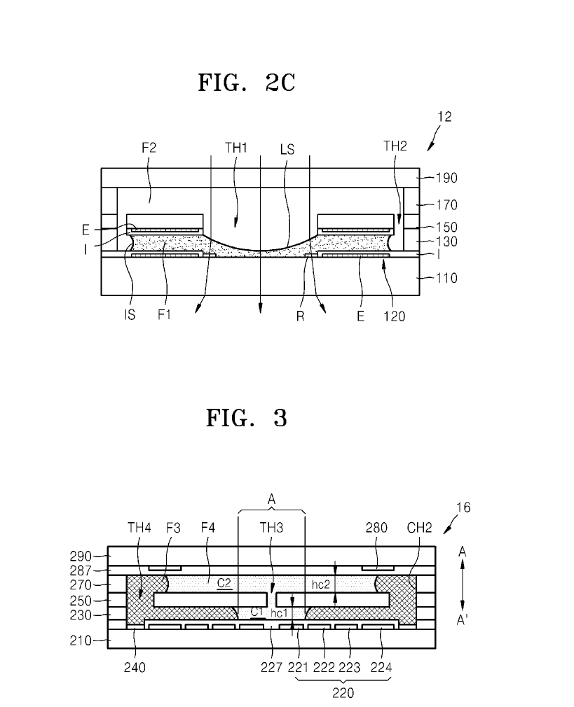

FIGS. 2A, 2B, and 2C are schematic cross-sectional views which illustrate a structure of the first liquid lens 12 of the zoom lens module illustrated in FIG. 1, according to an exemplary embodiment, wherein a lens surface having a curvature radius that varies according to a level of an applied voltage is illustrated.

Referring to FIGS. 2A, 2B, and 2C, the first liquid lens 12 is provided with a light-transmissive first lens fluid F1 and a light-transmissive second lens fluid F2 that is immiscible with the first lens fluid F1 in a first lens chamber CH1. An interface between the first lens fluid F1 and the second lens fluid F2 forms a lens surface which includes a first surface LS and a second surface IS that may facilitate a curvature variation of the lens surface. A lens electrode unit which creates an electric field for shifting the position of the second surface IS is installed in the first lens chamber CH1. In order for the interface between the first lens fluid F1 and the second lens fluid F2 to form the lens surface which includes the first surface LS and the second surface IS that may facilitate a curvature variation of the lens surface, a first intermediate lens substrate 150 which includes a first through-hole TH1 that defines a diameter of the lens corresponding to the lens surface and at least one second through-hole TH2 that forms a path of the second lens fluid F2 is disposed in the first lens chamber CH1. The shapes of the at least one second through-hole TH2 and the number of second through-holes are not limited to those illustrated in FIGS. 2A, 2B, and 2C.

A first lower lens substrate 110 and a first upper lens substrate 190 may be disposed below and above the first intermediate lens substrate 150, respectively. A spacer unit may be disposed between the first intermediate lens substrate 150 and the first upper lens substrate 190, and between the first intermediate lens substrate 150 and the first lower lens substrate 190. The spacer unit may include a first spacer 130 disposed between the first lower lens substrate 110 and the first intermediate lens substrate 150, and a second spacer 170 disposed between the first intermediate lens substrate 150 and the first upper lens substrate 190.

Each of the first lower lens substrate 110, the first intermediate lens substrate 150, and the first upper lens substrate 190 may be formed of a light-transmissive material.

The first lens fluid F1 and the second lens fluid F2 may include respective light-transmissive fluids that have different refractive indices. The first lens fluid F1 may include a non-polar liquid, and the second lens fluid F2 may include a gas or a non-polar liquid. A contact surface between the first lens fluid F1 and the second lens fluid F2 may include a hydrophobic coating layer, and in another exemplary embodiment, may be sealed with, for example, an elastic polymer-containing material which includes polydimethylsiloxane (PDMS).

As illustrated in FIGS. 2A, 2B, and 2C, the lens electrode unit may include a first lens electrode module 120 disposed on an upper surface of the first lower lens substrate 110 and including an electrode E which has a surface coated with an insulating material I; and a second lens electrode module 180 disposed on a lower surface of the first intermediate lens substrate 150 and including an electrode E which has a surface coated with an insulating material I. In another exemplary embodiment, the lens electrode unit may include only one of the first lens electrode module 120 and the second lens electrode module 180.

The electrodes E of the first lens electrode module 120 and the second lens electrode module 180 may be formed of a transparent conductive material. Examples of the transparent conductive material may include metal oxides, such as indium tin oxide (ITO) and indium zinc oxide (IZO); thin films in which metal nanoparticles of Au, Ag, or the like, for example, are dispersed; carbonaceous nanostructures, such as carbon nanotubes (CNT) and graphene; and conductive polymers, such as poly(3,4-ethylenedioxythiophene) (PEDOT), polypyrrole(PPy), and poly(3-hexylthiophene)(P3HT). A ground electrode R may be formed of any of the above-listed transparent conductive materials, and in another exemplary embodiment, may be formed as a metal thin film of Au, Ag, Al, Cr, or Ti, if light transmittance is not required, which depends on the location of the ground electrode R.

In the first liquid lens 12, a pressure exerted on the second surface IS may vary based on electric wetting, and a curvature of the first surface LS may be adjustable depending on a change in pressure acting on the second surface IS. Electric wetting refers to a phenomenon by which a contact angle of electrolyte droplets on an insulator-coated electrode varies when a voltage is applied to the electrolyte droplets. The contact angle may vary depending on interfacial tensions in a three-phase contact line (TCL) where a fluid, droplets, and an insulator meet. By using the electric wetting phenomenon, flow of fluids may be rapidly and effectively controllable at a low voltage, and transfer and control of fluids may be reversible.

According to the current exemplary embodiment, the liquid lens 12 includes the first lens electrode module 120 and the second lens electrode module 180, each including one electrode E, and the position of the second surface IS varies based on an adjustment of a voltage level applied to each electrode E. In particular, when a voltage is not applied, and when the second surface IS positioned as illustrated in FIG. 2A, the first surface LS, which forms the lens surface of the liquid lens 12, may have a maximum convex curvature which conforms to the position of the second surface IS. When a predetermined voltage is applied, as illustrated in FIG. 2B, the second surface IS may extend to opposite sides of the liquid lens 12, such that the first surface LS may have a reduced curvature. When a maximum voltage level is applied, as illustrated in FIG. 2C, the second surface IS may maximally extend toward opposite sides of the liquid lens 12, such that the first surface LS may have a concave curvature.

The first lower lens substrate 110 of the first liquid lens 12, as described above with reference to FIGS. 2A, 2B, and 2C, may be disposed to contact the aperture 16 of FIG. 1. The second liquid lens 14 may have the same structure as the first liquid lens 12. In an exemplary embodiment, in order to form the first liquid lens 12 and the second liquid lens 14 such that the respective lenses have different refractive indices, the second liquid lens 14 may include a first lens fluid F1 that differs from that the lens fluid used in the first liquid lens 12.

For example, the first liquid lens 12 may have a refractive index which is smaller than that the refractive index of the second liquid lens 14. The zoom lens module 10 of FIG. 1 may be installed in an endoscope system. When disposing the first liquid lens 12 to face a target image and the second liquid lens 14 toward an imaging unit (not shown), in order to prevent damage to the human body which would be caused by a failure of the zoom lens module 10, a saline solution which is harmless to the human body may be used as a solvent for both the first and second liquid lenses 12 and 14. Non-limiting examples of a solute for the first and second liquid lenses 12 and 14 may include NaCl, LiCl, and LiBr. The respective refractive index of each of the first and second liquid lenses 12 and 14 may be dependent on the concentration of the solute. In another exemplary embodiment, the first liquid lens 12 may contain an LiCl solution which has a solute concentration of about 15 or less, and the second liquid lens 14 may contain an LiCl solution which has a solute concentration of about 15 or greater.

The aperture 16 is disposed between the first and second liquid lenses 12 and 14, and may adjust the light transmission with a variable zoom magnification.

FIG. 3 is a schematic cross-sectional view which illustrates a structure of the aperture 16 of FIG. 1. Referring to FIG. 3, the aperture 16 may include a first channel C1 and a second channel C2 which is located above the first channel C1 and which is interconnected with the first channel C1, wherein the first channel C1 and the second channel C2 may contain a first aperture fluid F3 and a second aperture fluid F4, which flow therein, respectively. The first aperture fluid F3 and the second aperture fluid F4 may be immiscible with each other, and one of the first aperture fluid F3 and the second aperture fluid F4 may be light-transmissive, while the other one may have light-blocking ability. An aperture electrode unit may be provided for applying a voltage to generate an electric field depending on which an interfacial tension between the first aperture fluid F3 and the second aperture fluid F4 may be adjustable. The size of an opening A varies as the first aperture fluid F3 and the second aperture fluid F4 flow, such that a transmissivity of incident light may be varied correspondingly.

The first channel C1 and the second channel C2 constitute a single chamber, for example, a first aperture chamber CH2, with paths in peripheral and center regions connecting the first and second channels C1 and C2. A height hc2 of the second channel C2 may be equal to or greater than a height hc1 of the first channel C1.

In particular, the first channel C1 may be defined by a first lower aperture substrate 210, a first intermediate aperture substrate 250 disposed apart from the first lower aperture substrate 210 and including a first aperture through-hole TH3 in a center region and a second aperture through-hole TH4 in a peripheral region, and a first aperture spacer 230 disposed between the first lower aperture substrate 210 and the first intermediate aperture substrate 250 to define an internal space. The second channel C2 may be defined by the first intermediate aperture substrate 250, a first upper aperture substrate 290 disposed apart from the first intermediate aperture substrate 250, and a second aperture spacer 270 disposed between the first intermediate aperture substrate 250 and the first upper aperture substrate 290 to define an internal space. Although the first aperture through-hole TH3 appears to have a cross-sectional area which is smaller than the corresponding cross-sectional area of the second aperture through-hole TH4, this is exemplary, and the scope of the present disclosure is not limited thereto. The first lower aperture substrate 210, the first intermediate aperture substrate 250, and the first upper aperture substrate 290 may be formed of a light-transmissive material.

The first aperture fluid F3 may be a light-blocking or light-absorbing fluid, and may fill in the peripheral region of the first aperture chamber CH2. The first aperture fluid F3 may include a liquid metal or a polar liquid. In another exemplary embodiment, the first aperture fluid F3 may include a liquid metal, such as, for example, mercury (Hg), or a solution in which a dye that has an absorption wavelength appropriate for the liquid lens is contained. Non-limiting examples of the dye may include carbon black that absorbs a visible light wavelength range, near-infrared light-absorbing dyes having a maximum absorption wavelength of about 968 nm, and near-infrared light absorption dyes having a maximum absorption wavelength of about 1054 nm.

The second aperture fluid F4, which is a transparent fluid that is immiscible with the first aperture fluid F3, may be provided in the center region of the first aperture chamber CH2. Non-limiting examples of the fourth fluid F4 may include a gas or a non-polar liquid.

The first aperture fluid F3 and the second aperture fluid F4 may form fluidic interfaces in the first and second channels C1 and C2. The size of the opening A may be adjustable based on respective positions of these movable fluidic interfaces, as will be described below.

The aperture electrode unit may include a first aperture electrode module 220 which includes at least one electrode disposed on the first lower aperture substrate 210, and a second aperture electrode module 280 which includes at least one electrode disposed on the first upper aperture substrate 290. The at least one electrode of the first aperture electrode module 220 and the second aperture electrode module 280 may each be coated with an insulating material. In another exemplary embodiment, the first aperture electrode module 220 may be covered by a second dielectric layer 227, and the second aperture electrode module 280 may be covered by a third dielectric layer 287.

The first aperture electrode module 220 may include at least one electrode which is configured for digitally adjusting the size of the opening A. For example, the first aperture electrode module 220 may include, as illustrated in FIG. 3, a plurality of electrodes 221, 222, 223, and 224, which may form concentric annuli of different respective diameters. The second aperture electrode module 280 may also include at least one electrode. For example, the second aperture electrode module 280 may include one annular electrode, as illustrated in FIG. 3. The shapes of the electrodes and the number of electrodes that constitute the first aperture electrode module 220 and the second aperture electrode module 280 are not limited to those as illustrated in FIG. 3, and may vary differently.

A ground electrode unit 240 may be disposed at least on somewhere in the first aperture chamber CH2. In an exemplary embodiment, the ground electrode unit 240 may be disposed on the first lower aperture substrate 210 so as to contact the polar third fluid F3, as illustrated in FIG. 3.

The at least one electrode of the first aperture electrode module 220 and the second aperture electrode module 280 may be formed of a transparent conductive material. Examples of the transparent conductive material may include metal oxides, such as ITO and IZO; thin films in which metal nanoparticles of gold (Au), silver (Ag), or the like, for example, are dispersed; carbonaceous nanostructures, such as CNT and graphene; and conductive polymers, such as PEDOT, PPy, and P3HT.

The ground electrode unit 240 may not be required to be transparent due to its location, and may be formed as a metal thin film of, for example, gold (Au), silver (Ag), aluminum (Al), chromium (Cr), or titanium (Ti).

The size of the opening A of the aperture 50 may be varied by shifting of the interface between the first aperture fluid F3 and the second aperture fluid F4 toward a center direction or the opposite direction due to a pressure difference induced by a height difference between the first channel C1 and the second channel C2, a diameter difference between the first aperture through-hole TH3 and the second aperture through-hole TH4, and electric wetting.

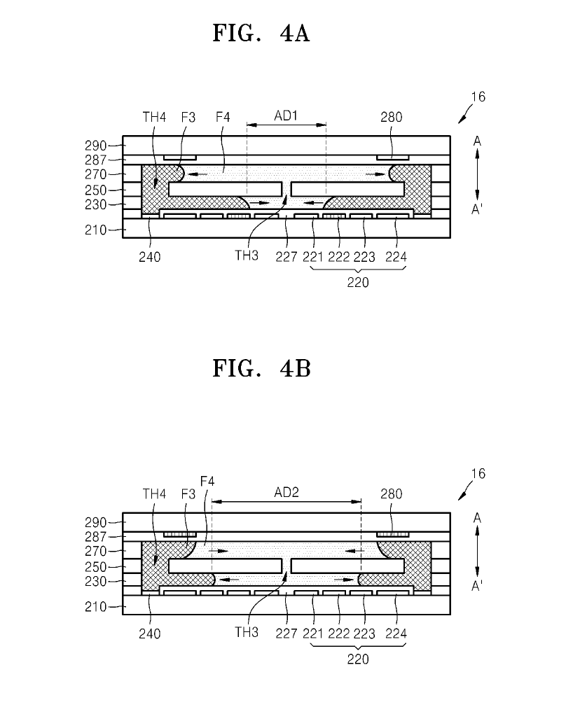

FIGS. 4A and 4B illustrate an adjustment of the light transmission of the aperture 16 of FIG. 3, based on different respective sizes AD1 and AD2 of the opening.

When an appropriate voltage is applied to one of the electrodes of the first aperture electrode module 220, an electromechanical force may be exerted at a three-phase contact line (TCL) on the activated driving electrode, for example, on the electrode 222, in which the first aperture fluid F3, the second aperture fluid F4, and the second dielectric layer 227 meet together, thereby shifting the first aperture fluid F3 in the first channel C1 to flow toward the center region, thereby reducing the size of the opening to have the diameter AD1, as illustrated in FIG. 4A.

When an appropriate voltage is applied to the second aperture electrode module 280, the first aperture fluid F3 in the second channel C2 may flow toward the center region, so that the TCL in the first channel C1 is pulled closer to the peripheral region, and thus the size of the opening is enlarged to have the diameter AD2, as illustrated in FIG. 4B.

In the exemplary embodiment in which the first aperture electrode module 220 includes a plurality of electrodes 221, 222, 223, and 224 that form concentric annuli, the size of the opening may be adjustable digitally by selectively activating the electrodes 221, 222, 223, and 224.

Although in the above exemplary embodiment the light-blocking or absorbing first aperture fluid F3 is polar, and the light-transmissive second aperture fluid F4 is non-polar, the polarity of the first aperture fluid F3 and the second aperture fluid F4 may be reversed. In particular, the first aperture fluid F3 may be non-polar, and the second aperture fluid F4 may be polar. Thus, in the latter instance, the opening and closing operation of the aperture 16 are opposite to the description provided with respect to the former instance. In particular, when a voltage is applied to the first aperture electrode module 220, the opening A may become larger. When a voltage is applied to the second aperture electrode module 280, the opening A may become smaller.

With the DOE lens array disposed between the first and second liquid lenses 12 and 14, the liquid lens may become small. A DOE lens array, which is an optical device that uses diffraction of light, may converge light into a single point via phase matching. In particular, this optical device, which has a small thickness, enables light reflected from an object to reach to an image via different paths but with the same phase, as though the light propagates through the same optical path in the zoom lens module 10.

The DOE lens array may include a combination of a plurality of DOE lenses.

Based on a movement of the DOE lens array in an optical axis direction, a focal distance of the DOE lenses may be adjusted.

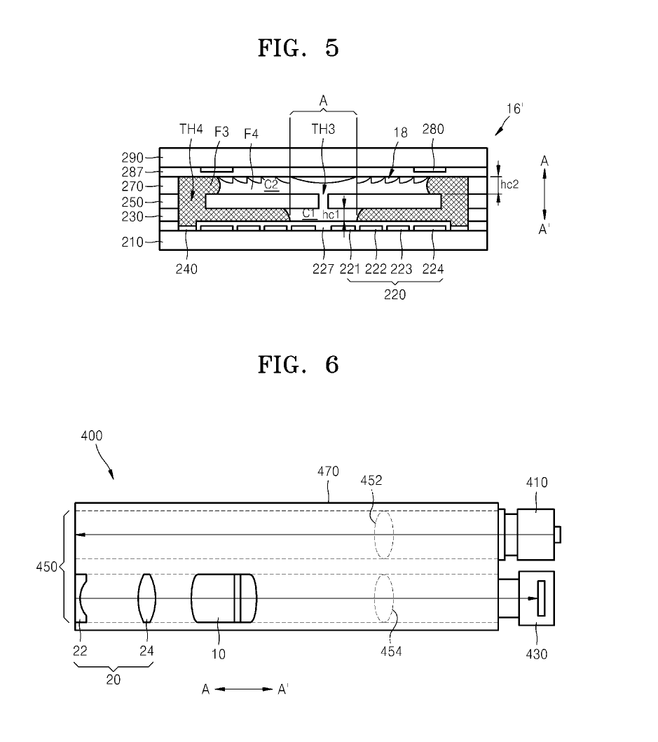

The DOE lens array may be disposed between the first liquid lens 12 and the aperture 16, or between the second liquid lens 14 and the aperture 16. In an exemplary embodiment, the DOE lens array may be disposed in the aperture 16. FIG. 5 illustrates an aperture 16′ which includes the DOE lens array 18, according to an exemplary embodiment. Referring to FIG. 5, the DOE lens array 18 may be disposed on the third dielectric layer 287 that covers the second aperture electrode module 280 in the aperture 16′. In an exemplary embodiment, the DOE lens array 18 may be disposed on the second dielectric layer 227 that covers the first aperture electrode module 220. The DOE lens array 18 may match the phase of light entering the opening consistently such that the light converges into a single point.

Endoscope systems obtain images of insides of internal organs or body cavities of a subject by being inserted into the body. The zoom lens module 10 described above may be installed in a small-diameter endoscope system.

FIG. 6 is a schematic illustration of an optical arrangement in an endoscope system 400, according to an exemplary embodiment. Referring to FIG. 6, the endoscope system 400 may include an illumination light providing unit 410 which provides illumination light, an imaging unit 430 which captures an image of a target, and a light transmission unit 450 which transmits the illumination light to the target and which transmits light reflected from the target to the imaging unit 430. The illumination light providing unit 410 and the imaging unit 430 may be configured to be detachably attached to the light transmission unit 450.

The illumination light providing unit 410 may provide illumination light to the target. The illumination light may have a pattern. The illumination light providing unit 410 may include an optical filter which blocks light having a wavelength corresponding to the pattern of the illumination light. The imaging unit 430 that captures an image of the target irradiated by the illumination light may include, for example, a complementary metal-oxide-semiconductor (CMOS) image sensor or a charge-coupled device (CCD) image sensor.

The light transmission unit 450 may include a first light transmission module 452 which transmits the illumination light to the target, and a second light transmission module 454 which transmits light reflected from the target to the imaging unit 430. Although in the exemplary embodiment illustrated in FIG. 6 the first light transmission module 452 and the second light transmission module 454 are provided as separate elements, the first light transmission module 452 may be configured to perform both its own and the function of the second light transmission module 454. The first light transmission module 452 and the second light transmission module 454 may be disposed in an insertion unit 470 of the endoscope system 400 that is thin and long so as to facilitate insertion into the body cavity of the target. The light transmission unit 450 may be configured as a waveguide that is able to pass through the insertion unit 470. For example, the light transmission unit 450 may be configured as a waveguide that can pass through from a leading end of the insertion unit 470 to a trailing end thereof.

A plurality of lenses 20 which are configured for guiding reflected light and for forming an image from the same may be disposed in the second light transmission module 454. A zoom lens module 10 which has a focal distance that is adjustable based on a change of at least one of its curvature and thickness may be disposed behind the lenses 20. The lenses 20 may be disposed in an order, beginning from near the target and proceeding outwardly. The lenses 20 may include a first lens 22 which has a negative refractive power and a second lens 24 which has a positive refractive power. The zoom lens module 10 may have the same structure as that described above with respect to one or more of the exemplary embodiments. For example, the zoom lens module 10 may include two liquid lenses which are disposed separate from one another, and an aperture disposed between the two liquid lenses. A focal distance of either of these liquid lenses may be adjusted based on a change of at least one of their respective curvatures and thicknesses, and the size of the opening of the aperture may be adjusted to transmit a constant amount of light even with a change in zoom magnification.

The illumination light providing unit 410 and the imaging unit 430 may be disposed separated from one another behind the insertion unit 470. For example, the illumination light providing unit 410 may be disposed behind the first light transmission module 452, and the imaging unit 430 may be disposed behind the second light transmission module 454.

If the endoscope system is used for capturing three-dimensional (3D) images, the endoscope system may include a plurality of imaging units which are used to acquire parallax images.

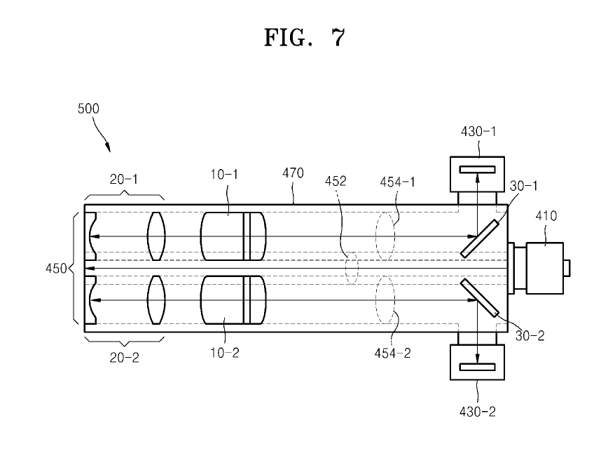

FIG. 7 is a schematic illustration of an optical arrangement in an endoscope system 500 which is configured for capturing three-dimensional images, according to another exemplary embodiment. Referring to FIG. 7, the endoscope system 500 may include an illumination light providing unit 410 which provides illumination light, a first imaging unit 430-1 which captures an image (hereinafter, a “left image”) of the target for the left eye, a second imaging unit 430-2 which captures an image (hereinafter, a “right image”) of the target for the right eye, and a light transmission unit 450 which transmits the illumination light to the target and which transmits light reflected from the target to the first and second image units 430-1 and 430 -2. The illumination light providing unit 410 and the first and second imaging units 430-1 and 430-2 may be configured to be detachably attached to the light transmission unit 450.

Because the endoscope system 500 of FIG. 7 is intended to be used for capturing three-dimensional images, the first and second imaging units 430-1 and 430-2 are disposed separated from one another in order to capture left and right images, respectively. The positioning of the first and second imaging units 430-1 and 430-2 behind the insertion unit 470 may increase the volume of the endoscope system 500. To avoid this, the first and second imaging units 430-1 and 430-2 may be disposed on opposite side ends of the insertion unit 470, respectively.

The light transmission unit 450 may include a first light transmission module 452 which transmits illumination light to the target, a second light transmission module 454-1 which transmits light reflected from the target to the first imaging unit 430-1, and a third light transmission module 454-2 which transmits light reflected from the target to the second imaging unit 430-2.

The first, second, and third light transmission modules 452, 454-1, and 454-2 may be configured as waveguides which are able to pass through the insertion unit 470. For example, the first light transmission module 452 may be configured as a waveguide that can pass through from a leading end of the insertion unit 470 to a trailing end thereof. The second and third light transmission modules 454-1 and 454-2 may be configured as waveguides that can pass through from the leading end of the insertion unit 470 to the side ends thereof at the trailing end. Therefore, the first and second imaging units 430-1 and 430-2 may be disposed on the side ends of the insertion unit 470 behind the second light transmission module 454-1 and the third light transmission module 454-2, respectively. As a result of being disposed on the side ends of the insertion unit 470, the first and second imaging units 430-1 and 430-2 may each include a curved region, and thus the second and third light transmission modules 454-1 and 454-2 may each include a curved region, and thus, first and second reflecting units 30-1 and 30-2 may be further disposed at the curved regions of the first and second imaging units 430-1 and 430-2, respectively, to reflect light incident thereon. The first and second reflecting units 30-1 and 30-2 may be implemented, for example, as mirrors.

A plurality of lenses which are configured for guiding reflected light and for forming an image from the same may be disposed in the second and third light transmission modules 454-1 and 454-2. These lenses may be disposed in the second and third light transmission modules 454-1 and 454-2 near a leading end thereof adjacent to the target, and may include a plurality of lenses 20-1 and 20-2. Zoom lens modules 10-1 and 10-2, for which a focal distance may be adjusted based on a change of at least one of a respective curvature and a respective thickness of a liquid lens contained therein, may be disposed behind the lens 20-1 and the lens 20-2, respectively. The lenses 20-1 and 20-2 and the zoom lens modules 10-1 and 10-2 of FIG. 7 are the same as or similar to the lenses 20 and the zoom lens module 10 of FIG. 6, respectively. The two zoom lens modules 10-1 and 10 -2 may be integrated into one body. In another exemplary embodiment, the first liquid lens, the aperture, and the second liquid lens of the zoom lens module 10-1 may be integrated with those of the zoom lens module 10-2, respectively.

Although in the exemplary embodiment illustrated in FIG. 7 the first, second, and third light transmission modules 452, 454-1, and 454-2 are provided as separate elements, the scope of the present disclosure is not limited thereto. In another exemplary embodiment, the light transmission unit 450 may not include the first light transmission module 452, and instead, may provide illumination light to the target by employing the second light transmission module 454-1 or the third light transmission module 454-2.

Although in the exemplary embodiment illustrated FIG. 7 the first imaging unit 430-1 and the second imaging unit 430-2 are disposed at the side ends of the insertion unit 470, the scope of the present disclosure is not limited thereto. In another exemplary embodiment, the first imaging unit 430-1 and the second imaging unit 430-2 may be disposed in the insertion unit 470.

As described above, according to the one or more of the above-described exemplary embodiments, with the use of a zoom lens module in an endoscope system which is configured for capturing three-dimensional images, sharper three-dimensional images may be obtained.

According to the one or more exemplary embodiments, a zoom magnification of the zoom lens module may be adjustable based on a change of at least one of a curvature and a thickness of its liquid lens, so that the zoom lens module may be small in size.

Even with a change in zoom magnification, an aperture which is disposed between a plurality of liquid lenses and that is configured to adjust an amount of light transmission ensures acquisition of high-quality images.

An endoscope system having a zoom function may be implemented by using the above-described small zoom lens module.

It should be understood that the exemplary embodiments described herein should be considered in a descriptive sense only and not for purposes of limitation. Descriptions of features or aspects within each embodiment should typically be considered as available for other similar features or aspects in other embodiments.

1. A zoom lens module comprising:
a first liquid lens;
a second liquid lens disposed separated from the first liquid lens; and
an aperture disposed between the first and the second liquid lenses,
wherein a respective focal distance of each of the first liquid lens and the second liquid lens is adjustable based on a change of at least one of a respective curvature thereof and a respective thickness thereof.

2. The zoom lens module of claim 1, further comprising a diffractive optical element (DOE) lens array disposed at least one of between the first liquid lens and the aperture, and between the aperture and the second liquid lens.

3. The zoom lens module of claim 2, further comprising a dielectric layer on the DOE lens array.

4. The zoom lens module of claim 1, wherein at least one of the first and second liquid lenses has a curvature radius which is less than or approximately equal to 2.5 mm.

5. The zoom lens module of claim 1, wherein an interval between the first liquid lens and the second liquid lens has a length which is less than or approximately equal to 2.5 mm.

6. The zoom lens module of claim 1, wherein at least one of the first and second liquid lenses comprises:
a first lens fluid;
a second lens fluid that is immiscible with the first lens fluid;
a first lens chamber which contains the first lens fluid and the second lens fluid;
a first surface which functions as an interface between the first lens fluid and the second lens fluid to form a lens surface;
a second surface which functions as an interface between the first lens fluid and the second lens fluid that facilitates a change in a curvature of the lens surface; and
a first lens electrode unit which shifts a position of the second surface to effect the change in the curvature of the lens surface.

7. The zoom lens module of claim 6, wherein each of the first lens fluid and the second lens fluid is light-transmissive.

8. The zoom lens module of claim 6, further comprising a first intermediate lens substrate provided in the first chamber, the first intermediate lens substrate including a first through-hole which defines a diameter of a lens corresponding to the lens surface and a second through-hole which defines a path of the second lens fluid.

9. The zoom lens module of claim 8, further comprising:
a first lower lens substrate disposed below the first intermediate lens substrate;
a first upper lens substrate disposed above the first intermediate lens substrate; and
a first spacer unit disposed between the first lower lens substrate and the first intermediate lens substrate, and
a second spacer unit disposed between the first intermediate lens substrate and the first upper lens substrate.

10. The zoom lens module of claim 6, wherein the first lens electrode unit comprises at least one electrode coated with an insulating material.

11. The zoom lens module of claim 1, wherein the aperture comprises:
a first aperture fluid;
a second aperture fluid that is immiscible with the first aperture fluid, wherein one of the first aperture fluid and the second aperture fluid is light-transmissive and an other of the first aperture fluid and the second aperture fluid is formed of a light-blocking material;
a first aperture chamber which contains the first aperture fluid and the second aperture fluid; and
a first aperture electrode unit which adjusts a size of an opening through which light passes by shifting a position of an interface between the first aperture fluid and the second aperture fluid.

12. The zoom lens module of claim 11, wherein the first aperture chamber comprises:
a channel region which corresponds to a range of the size of the opening that is adjustable by shifting the position of the interface between the first aperture fluid and the second aperture fluid; and
a reservoir region which stores each of the first and second aperture fluids such that each of the first and second aperture fluids is arranged to move into the channel region based on a shift in the position of the interface between the first aperture fluid and the second aperture fluid.

13. The zoom lens module of claim 11, wherein the first aperture chamber comprises:
a first lower aperture substrate which contains the first aperture electrode unit;
a first intermediate aperture substrate disposed facing toward and separated from the first lower aperture substrate; and
a first upper aperture substrate disposed facing toward and separated from the first intermediate aperture substrate.

14. The zoom lens module of claim 13, wherein the first intermediate aperture substrate comprises a through-hole in a center region thereof.

15. The zoom lens module of claim 14, wherein the one of the first aperture fluid and the second aperture fluid that is light-transmissive is provided in a center region of the first aperture chamber, and the other of the first aperture fluid and the second aperture fluid that is formed of the light-blocking material is provided in a peripheral region of the first aperture chamber which peripheral region surrounds the center region.

16. The zoom lens module of claim 11, wherein the first aperture chamber comprises:
a first channel; and
a second channel disposed on the first channel, the second channel being interconnected with the first channel,
wherein a range of the size of the opening is defined by a corresponding range of shifts in the position of the interface between the first aperture fluid and the aperture fluid within each of the first and second channels.

17. An endoscope system comprising:
an illumination light providing unit which provides illumination light to a target;
an imaging unit which captures an image of the target; and
a light transmission unit which comprises the zoom lens module according to claim 1, and which transmits the illumination light to the target and which transmits light reflected from the target to the imaging unit.

18. The endoscope system of claim 17, further comprising an insertion unit within which the light transmission unit is installed and which is insertable into a body cavity.

19. The endoscope system of claim 18, wherein the light transmission unit includes a waveguide.

20. The endoscope system of claim 17, wherein the light transmission unit comprises:
a first light transmission module which transmits the illumination light to the target; and
a second light transmission module which comprises the zoom lens module and which transmits the light reflected from the target to the imaging unit.

21. The endoscope system of claim 17, wherein the imaging unit comprises:
a first imaging unit which captures at least a first parallax image of the target; and
a second imaging unit which is disposed separated from the first imaging unit and which captures at least a second parallax image of the target,
wherein the at least first parallax image and the at least second parallax image are used for creation of at least one three-dimensional image.

22. The endoscope system of claim 21, wherein the light transmission unit comprises:
a first light transmission module which comprises the zoom lens module and which transmits a first part of the light reflected from the target to the first imaging unit; and
a second light transmission module which comprises the zoom lens module and which transmits a second part of the light reflected from the target to the second imaging unit.

23. The endoscope system of claim 22, wherein at least one of the first and second light transmission modules comprises at least one curved region in which a reflecting unit for reflecting light incident on the curved region is disposed.
